(19) United States
(12) Patent Application Publication
(10) Pub. No.: US 2023/0390170 A1
(43) Pub. Date: Dec. 7, 2023

(54) CONSUMER PRODUCTS CONTAINING PRO-FRAGRANCES

(71) Applicant: Firmenich SA, Satigny (CH)

(72) Inventors: Murray OSBORNE, Satigny (CH); Philippe GAUMANN, Satigny (CH)

(21) Appl. No.: 18/246,513

(22) PCT Filed: Sep. 24, 2021
(86) PCT No.: PCT/EP2021/076289

§ 371 Date: Mar. 23, 2023

(30) Foreign Application Priority Data
Sep. 24, 2020 (EP) 20198175.0

(51) Int. Cl. A61K 8/36; C11D 11/00; C11D 3/00; C11D 3/50; C11D 3/20; C11D 3/34; A61Q 5/02; A61K 8/46

(52) U.S. Cl. CPC A61K 8/36; C11D 11/0017; C11D 3/0015; C11D 3/50; C11D 3/2079; C11D 3/3427; A61Q 5/02; A61K 8/46

(57) ABSTRACT

The invention relates to a perfumed consumer product a) an active base; b) at least one Zinc salt of formula Zn(RCOO)₂ (I) wherein R is a hydrogen atom or a linear C₁-C₂₀ alkyl or linear C₂-C₂₀ alkenyl group; c) at least one sulfur-containing pro-fragrance compound; and d) one or more perfuming co-ingredients. The perfumed consumer product compositions can be advantageously used in methods of treatment of fabric and household surfaces, to impart thereto a fresh, odor stable and long-lasting fragrance. They also show a stable out of the bottle odor, without any off-odor notes, independently of the duration of storage of the bottled liquid products.

### TECHNICAL FIELD

The present invention relates to perfumery. More particularly, it concerns a perfumed consumer product comprising a combination of ingredients from four different classes and which are capable of imparting a long-lasting odor and better freshness to surfaces such as textiles or hard surfaces, as well as having improved odor stability out of the bottle and after prolonged storage.

### BACKGROUND

Consumers want products that leave a clean and long-lasting freshness odor impression on their textiles and other surfaces treated with cleaning or softening products. Several technologies have been used in the past to achieve this effect, in particular fragrances have been encapsulated prior to incorporation in the treating product, or the experts have resorted to the use of so-called pro-fragrances, i.e. molecules that typically do not themselves impart an odor, but which are capable of releasing an odorant over a certain period of time under use conditions.

As a result, the consumer's perception and preference of the cleaning product can be dictated by the odor that it exhales upon opening of the bottle and by the residual odor that such a product is capable of imparting to the treated surfaces.

Amongst the prior known solutions to prolong the freshness and cleanliness effect of textiles treated with detergents and/or fabric softeners containing pro-fragrances, one can cite in particular the compounds and compositions that are described for example in U.S. Pat. No. 7,723,286, WO 2008/154765 or EP3386939. These prior art documents describe compounds which are capable of releasing one or more ingredients imparting an odor to fabrics or other treated surfaces, the latter having been subjected to the action of a consumer product comprising such compounds. WO2012113746 reports a composition, in particular a liquid fabric softener or all-purpose cleaner, comprising a liquid base, a sulfur-containing compound, at least one sulfur-containing pro-fragrance compound and one or more perfuming co-ingredients in order to improve the long-lastingness of the perceived odor while maintaining an acceptable odour quality after storage. However, the use of the sulfur-containing compound reported in WO2012113746 in consumer product has been limited.

So there is a need to develop novel composition allowing to benefit the long-lastingness provided by the sulfur-containing pro-fragrance compound while avoiding any formation of off-note or premature degradation of the sulfur-containing pro-fragrance compound.

The present invention brings a new and advantageous contribution to this field by providing products or compositions, namely fabric softeners and all-purpose cleaners, wherein the compounds described in the above-cited prior art documents are combined with particular agents capable of stabilizing the odor impact of the product and to improve its effectiveness to impart a long-lasting, clean and fresh odor to the fabrics or other surfaces treated with such products.

### BRIEF DESCRIPTION OF THE DRAWINGS

FIG. 1: Quantification of H₂S in a fabric softener base comprising different Zinc salts

### DESCRIPTION OF THE INVENTION

Surprisingly, it has now been discovered that the addition of a Zinc salt of formula (I) to a perfumed consumer product comprising an active base, at least one sulfur-containing pro-fragrance compound and one or more perfuming co-ingredients allows improving the odor stability and allows limiting the generation of off-note. Only zinc salts of formula (I) allows scavenging efficiency the H₂S generated and responsible of the degradation of odor over time.

So a first object of the present invention is therefore a perfumed consumer product comprising:
a) an active base;
b) at least one Zinc salt of formula

Zn(RCOO)₂ (I)
wherein R is a hydrogen atom or a linear C₁-C₂₀ alkyl or linear C₂-C₂₀ alkenyl group;
c) at least one sulfur-containing pro-fragrance compound; and
d) one or more perfuming co-ingredients.

For the sake of clarity, “perfumed consumer product” is meant to designate a consumer product which is expected to deliver among different benefits a pleasant perfuming effect to the surface or space to which it is applied (e.g. skin, hair, textile, paper or home surface) or in the air (air-freshener, deodorizer etc). In other words, a perfumed consumer product according to the invention is a manufactured product which comprises a functional formulation also referred to as “base”, together with benefit agents, corresponding to the desired consumer product, and an olfactive effective amount of one or more perfuming ingredients. For the sake of clarity, said perfumed consumer product is a non-edible product.

The terms “alkenyl” is understood as comprising 1, 2 or more olefinic double bonds, particularly one double bond

The nature and type of the constituents of the perfumed consumer product do not warrant a more detailed description here, which in any case would not be exhaustive, the skilled person being able to select them on the basis of his general knowledge and according to the nature and the desired effect of said product.

Non-limiting examples of suitable perfumed consumer products include a perfume, such as a fine perfume, a splash or eau de parfum, a cologne or a shave or after-shave lotion; a fabric care product, such as a liquid or solid detergent, a fabric softener, a liquid or solid scent booster, a fabric refresher, an ironing water, a paper, a bleach, a carpet cleaner, a curtain-care product; a body-care product, such as a hair care product (e.g. a shampoo, a coloring preparation or a hair spray, a color-care product, a hair shaping product, a dental care product), a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner.

By active base, it is meant here ingredient(s) capable of imparting a benefit associated with the consumer product such as texture, moisturizing agent, etc. . . . . The active base comprises ingredients that are common in the perfumed consumer products. The active base of a perfumed consumer product depend on the nature of the perfumed consumer product and can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

According to specific embodiments of the perfumed consumer product, the latter consist of a mixture of the above-cited components. By a perfumed consumer product “consisting of” it is understood here a composition which contains essentially the four components cited above, together with insignificant, i.e. not more than 2% by weight, and preferably not more than 1% weight, relative to the weight of the perfumed consumer product, of any other components and wherein the latter do not significantly affect the cleaning, softening and/or perfuming properties and activity of the perfumed consumer product.

According to any embodiments of the invention, the perfumed consumer product of the invention are characterized by a pH of 1 or more. Particularly, the perfumed consumer product of the invention has a pH comprised between 1 and 12, or between 1 and 8. Even more particularly, the perfumed consumer product of the invention has a pH comprised between 1 and 6.

According to a particular embodiment, the invention's perfumed consumer product is in the form of a personal care, a home care or fabric care consumer product. The component a) as defined above is typically an active base comprising ingredients that are common in the personal, home or fabric care consumer products, in particular shower gel, shampoo, soap, fabric detergents or softeners and all-purpose cleaners. The main functional components of such bases are surfactants and/or softener components capable of cleaning and/or softening fabrics and/or textiles of varied nature, such as clothes, curtain fabrics, carpet and furniture fabrics, etc., or other home surfaces, skin or hair, and typically used in a large amount of water or water-based solvents. These are therefore formulations wherein the amount of water is typically comprised between 50 and 99% by weight of the active base with the exception of soap or solid detergent wherein the amount of water is at most 20%. The term “base” is used here in the sense of the main component of the composition according to the invention and not in the sense of a liquid having a basic pH.

A more detailed description of such fabric cleaning and/or softening bases is not warranted here, many descriptions of current liquid bases can be found in the cleaner/fabric softener's patent and other pertinent literature, such as for example the textbook of Louis Ho Tan Tai, “Détergents et Produits de Soins Corporels, Chapters 1 to 7 in particular, Dunod, Paris, 1999, or any other similar and/or more recent textbooks pertaining to the art of liquid softener and all-purpose cleaners formulations. A patent publication, WO 2010/105873, is also cited by way of example, in as much as it describes typical current ingredients, other than perfumes, of such liquid products, particularly in pages 9 to 21. Of course, many other examples of liquid cleaner and/or fabric softener bases can be found in the literature. Any such liquid base, namely liquid fabric cleaner or conditioner and/or all-purpose cleaner, can be used in the here-described compositions.

According to any embodiments of the invention, component a) typically represents at least 85%, even 90% by weight, and may represent up to 99.95% by weight, of the total weight of the composition according to the invention, typically from 95% to 99.9% of the total weight of the invention's compositions, and more preferably from 98% to 99.8% of the total weight of the product.

According to a particular embodiment of the invention, the invention's perfumed consumer product is a liquid fabric softener wherein component a) is a fabric softener active base in amount comprised between 85 and 100% by weight, based on the total weight of the perfumed consumer product. The fabric softener active base may comprise dialkyl quaternary ammonium salts, dialkyl ester quaternary ammonium salts, Hamburg esterquat, triethanolamine quat, silicones and mixtures thereof. Optionally, component a) of the composition may further comprise a viscosity modifier in an amount comprised between 0.05 and 1% by weight, based on the total weight of the liquid base; preferably chosen in the group consisting of calcium chloride.

According to a particular embodiment of the invention, the invention's perfumed consumer product is an all-purpose cleaner wherein component a) of the composition is an all-purpose cleaner active base in amount comprised between 85 and 100% by weight, based on the total weight of the perfumed consumer product. The all-purpose active base may comprise linear alkylbenzene sulfonates (LAS) in an amount comprised between 1 and 2%, nonionic surfactant in an amount comprised between 2 and 4% and acid such as citric acid in an amount comprised between 0.1 and 0.5%.

According to a particular embodiment of the invention, the invention's perfumed consumer product is a liquid detergent wherein component a) is a liquid detergent active base in amount comprised between 85 and 100% by weight, based on the total weight of the perfumed consumer product. The liquid detergent active base may comprise anionic surfactant such as alkylbenzenesulfonate (ABS), linear alkylbenzene sulfonates (LAS), secondary alkyl sulfonate (SAS), primary alcohol sulfate (PAS), lauryl ether sulfate (LES), sodium lauryl ether sulfate (SLES), methyl ester sulfonate (MES); nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides; or mixtures thereof.

According to a particular embodiment of the invention, the invention's perfumed consumer product is a solid detergent wherein component a) is a solid detergent active base in amount comprised between 85 and 100% by weight, based on the total weight of the perfumed consumer product. The solid detergent active base may comprise at least one surfactant chosen in the group consisting of anionic, nonionic, cationic, zwiterionic surfactant and mixtures thereof. The surfactant in the solid detergent active base is preferably chosen in the group consisting of linear alkene benzene sulphonate (LABS), sodium laureth sulphate, sodium lauryl ether sulphate (SLES), sodium lauryl sulphate (SLS), alpha olefin sulphonate (AOS), methyl ester sulphonates (MES), alkyl polyglyucosides (APG), primary alcohol ethoxylates and in particular lauryl alcohol ethoxylates (LAE), primary alcohol sulphonates (PAS), soap and mixtures thereof. The solid detergent active base may comprise a further component, commonly used in powder detergent consumer product, selected from the group consisting of bleaching agents such as TAED (tetraacetylethylenediamine); buffering agent; builders such as zeolites, sodium carbonate or mixture thereof; soil release or soil suspension polymers; granulated enzyme particles such as cellulase, lipase, protease, mannanase, pectinase or mixtures thereof; corrosion inhibitor; antifoaming; sud suppressing agents; dyes; fillers such as sodium silicate, sodium sulfate or mixture thereof; source of hydrogen peroxide such as sodium percarbonate or sodium perborate; and mixtures thereof.

According to a particular embodiment of the invention, the invention's perfumed consumer product is shampoo or a shower gel wherein component a) is a shampoo or shower gel active base in amount comprised between 85 and 100% by weight, based on the total weight of the perfumed consumer product. The shampoo shower gel active base may comprise sodium alkylether sulfate, ammonium alkylether sulfates, alkylamphoacetate, cocamidopropyl betaine, cocamide MEA, alkylglucosides and aminoacid based surfactants.

According to a particular embodiment of the invention, the invention's perfumed consumer product is a soap bar wherein component a) is a soap active base in amount comprised between 85 and 100% by weight, based on the total weight of the perfumed consumer product. The soap bar active base may comprise salt of a weak acid, typically, a salt of weak acid, which may be a fatty acid and strong base like sodium hydroxide.

According to any embodiments of the invention, component b) of the perfumed consumer product according to the invention is a Zinc salt of formula

Zn(RCOO)₂ (I)

wherein R may be a hydrogen atom or a linear C₁-C₁₈ alkyl or linear C₂-C₁₈ alkenyl group. Particularly, R may be a hydrogen atom or a linear C₁-C₁₈ alkyl group. Particularly, R may be a hydrogen atom or a linear C₁-C₁₅ alkyl group. Particularly, R may be a hydrogen atom or a linear C₁-C₁₀ alkyl group. Particularly, R may be a hydrogen atom or a linear C₁-C₈ alkyl group. Particularly, R may be a hydrogen atom or a linear C₁-C₆ alkyl group. Particularly, R may be a hydrogen atom or a C₁-C₄ linear alkyl group. Particularly, R may be a hydrogen atom or a C₁-C₃ linear alkyl group. Even more particularly, R may be a methyl, an ethyl or a heptadecyl group. Particularly, the zinc salt may be selected from the group consisting of Zinc acetate and Zinc stearate.

Component b) may be present in the perfumed consumer product of the invention at a weight concentration of 0.01% or more, relative to the total weight of the perfumed consumer product. It can be present at a concentration up to 5% of the total weight of the perfumed consumer product. Particularly, the concentration of Zinc salt in the perfumed consumer product may be comprised between 0.01 and 3% based on the total weight of the perfumed consumer product. Even more particularly, the concentration of Zinc salt in the perfumed consumer product may be comprised between 0.1 and 3% based on the total weight of the perfumed consumer product.

The compositions of the invention contain a pro-fragrance as component c) thereof. By a “pro-fragrance” it is understood here a component that is one or more of the compounds described in the prior art cited above, i.e. U.S. Pat. No. 7,723,286, WO 2014/202591 and/or WO 2008/154765. Such compounds, although non-odorant as such, have the ability to release fragrant molecules under use/application conditions, i.e. upon application of the compositions according to the invention. The compositions may contain one or several such compounds, the latter allowing the controlled release of a variety of different odor imparting substances, which may be an advantage over slowly releasing just one fragrance ingredient as will happen if just one pro-fragrance compound is used.

According to specific embodiments of any one of the compositions of the invention, the sulfur-containing pro-fragrance compound is a compound of formula

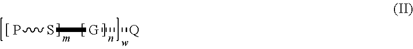

wherein:
a) w represents an integer from 1 to 10000;
b) n represents 1 or 0;
c) m represents an integer from 1 to 6;
d) P represents a hydrogen atom or a radical susceptible of generating an odoriferous α,β-unsaturated ketone, aldehyde or carboxylic ester and is represented by the formula

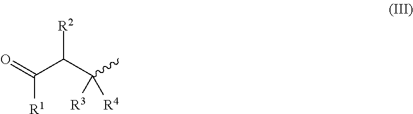

in which the wavy line indicates the location of the bond between said P and S; R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups; and with the proviso that at least one of the P groups is of the formula (III) as defined hereinabove;
e) G represents a multivalent radical (with a m+1 valence) derived from cyclic, linear, alicyclic or branched alkyl, cyclic, linear, alicyclic or branched alkenyl, phenyl, alkylphenyl or alkenylphenyl hydrocarbon radical having from 1 to 22 carbon atoms, said hydrocarbon radical being possibly substituted and containing from 1 to 10 functional groups selected from the group consisting of halogens, alcohols, ethers, esters, ketones, aldehydes, carboxylic acids, thiols, thioethers, amines, quaternary amines and amides; and
f) Q represents a hydrogen atom (in which case w=1 and n=1), or represents a polymer or co-polymer selected from the group consisting of poly(alkylimine)s, peptides (e.g. lysine) or polysaccharides selected from the group consisting of cellulose, cyclodextrins and starches, or cationic quaternised silicon polymers, or still a polymer or random co-polymer derived from monomeric units selected from the group consisting of the formulae A) to C):

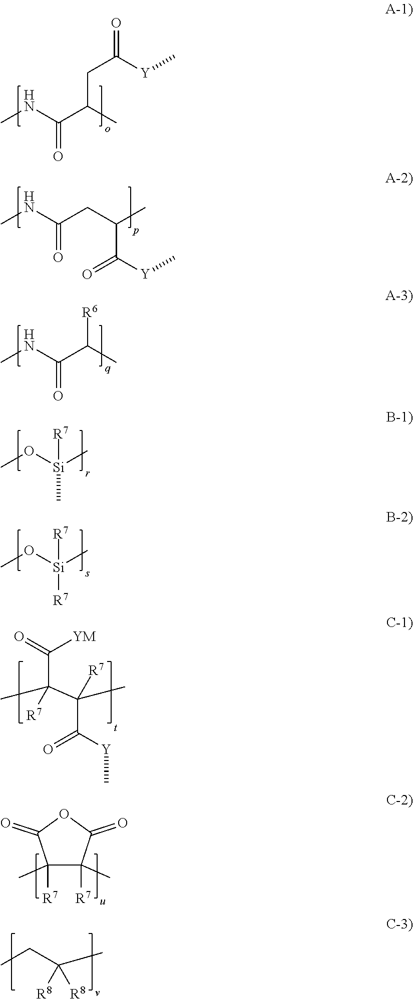

wherein the hatched lines indicate the location of the bond between said monomeric unit and G;
Y represents an oxygen or sulfur atom or a NR⁷ group;
o, p, q, r, s, t, u and v all represent independent of each other fractions between 0 and 1, with o+p+q=1, r+s=1 and t+u+v=1 and with the proviso that either o or p, as well as r and t are not equal to 0;
R⁶ represents a hydrogen atom or a side chain from a natural or unnatural amino acid, such as glycine, alanine, phenylalanine, arginine, histidine, lysine, aspartic acid, glutamic acid, cysteine, methionine, glutamine, asparagine, threonine, serine, leucine, isoleucine, valine, tyrosine or tryptophan;
R⁷ represents, simultaneously or independently, a hydrogen atom or a C₁-C₁₆ hydrocarbon group;
R⁸ represents, simultaneously or independently of each other
   a hydrogen or halide atom;
   a C₁-C₆ hydrocarbon group optionally comprising from 1 to 4 heteroatoms selected from the group consisting of oxygen and sulfur atoms;
   a carboxylic group of formula COOR*, wherein R* represents a hydrogen atom, a C₁-C₆₀ alkyl or alkenyl group optionally comprising from 1 to 30 oxygen atoms;
   a OR⁷ group or a COR⁷ group; or
   a pyrrolidone unit, connected by the nitrogen atom; and
M represents a hydrogen atom, an alkali or earth alkali metal ion.

As “odoriferous α,β-unsaturated ketone, aldehyde or carboxylic ester”, the expression used in the definition of P, it is understood an α,β-unsaturated ketone, aldehyde or carboxylic ester, which is recognized by a skilled person as being used in perfumery as perfuming ingredient. In general, said odoriferous α,β-unsaturated ketone, aldehyde or carboxylic ester is a compound having from 8 to 20 carbon atoms, or even more preferably between 10 and 15 carbon atoms.

Similarly, it is not possible to provide an exhaustive list of the currently known odoriferous compounds, which can be used in the synthesis of the invention compounds defined hereinabove and subsequently be released. However, the following can be named as preferred examples: alpha-damascone, beta-damascone, gamma-damascone, delta-damascone, alpha-ionone, beta-ionone, gamma-ionone, delta-ionone, beta-damascenone, 1-[6-ethyl-2,6-dimethyl-3-cyclohexen-1-yl]-2-buten-1-one, 3-methyl-5-propyl-2-cyclohexen-1-one, 2-methyl-5-(1-propen-2-yl)-2-cyclohexen-1-one, 2,5-dimethyl-5-phenyl-1-hexen-3-one, 1-(5,5-dimethyl-1-cyclohexen-1-yl)-4-penten-1-one, 3,7-dimethylocta-2,6-dienal, 8-methyl-alpha-ionone or 10-methyl-alpha-ionone, 2-octenal, 1-(2,2,3,6-tetramethylcyclohexyl)but-2-en-1-one, 4-(2,2,3,6-tetramethylcyclohexyl)but-3-en-2-one, 2-cyclopentadecen-1-one, 4,4a-dimethyl-6-(1-propen-2-yl)-4,4a,5,6,7,8-hexahydro-2(3H)-naphthalenone, (E)-3-phenylprop-2-enal (cinnamic aldehyde), 2,6,6-trimethylspiro[bicyclo[3.1.1]heptane-3,1′-cyclohexan]-2′-en-4′-one, ethyl 2,4-deca-dienoate, ethyl 2-octenoate, methyl 2-nonenoate, ethyl 2,4-undecadienoate, 4-methylpent-3-en-2-one, oct-2-en-4-one, and methyl 5,9-dimethyl-2,4,8-decatrienoate.

In a particular embodiment, P represents a radical selected from the group consisting of formulae (P-1) to (P-14), in the form of any one of its isomers:

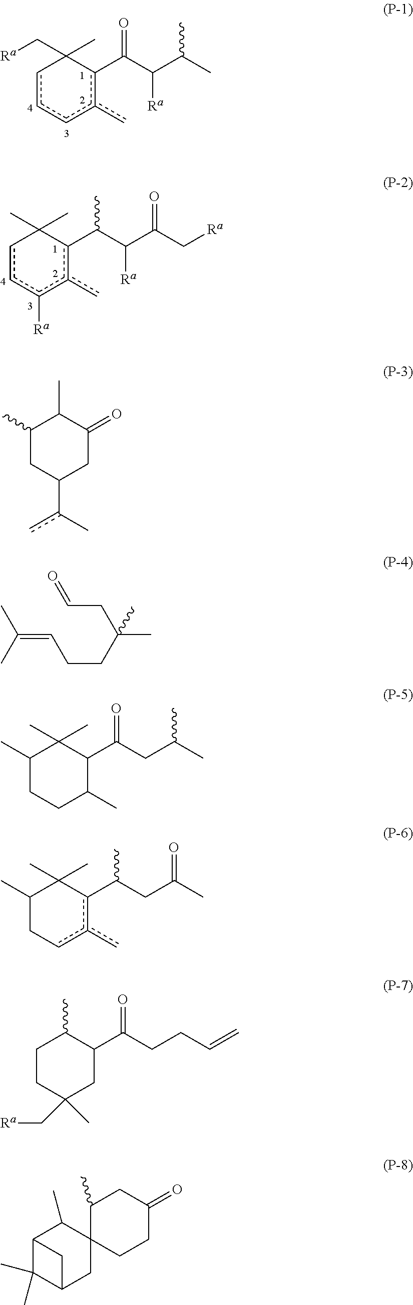

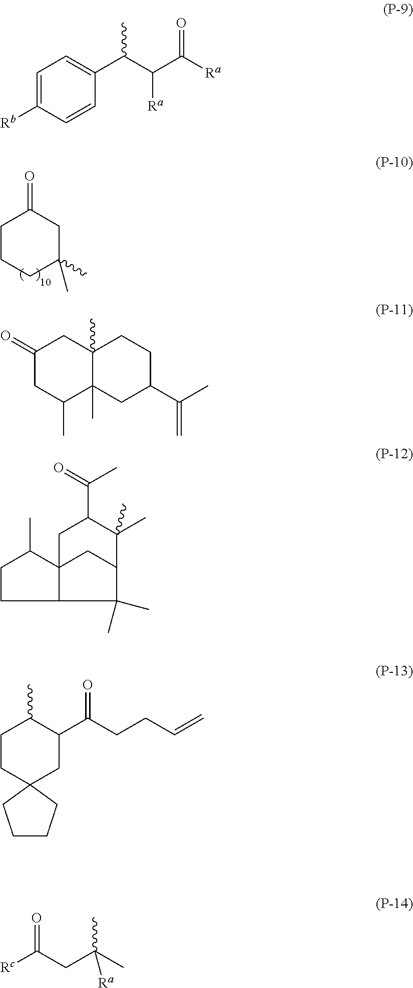

in which formulae the wavy lines have the meaning indicated above and the dotted lines represent a carbon-carbon single or double bond, Ra being a hydrogen atom or a methyl group and R^{b} representing a hydrogen atom, a hydroxyl or methoxy group or a C₁-C₄ linear or branched alkyl group and R representing a hydrogen atom or a C₁-C₄ linear or branched alkyl group.

For the sake of clarity, by the expression “the dotted lines represent a carbon-carbon single or double bond”, or the similar, it is meant the normal meaning understood by a person skilled in the art, i.e. that the whole bonding (solid and dotted line) between the carbon atoms connected by said dotted line is a carbon-carbon single or double bond, provided that 2 adjacent dotted lines are not both carbon-carbon double bond.

In a particular embodiment, P represents a radical selected from the group consisting of formulae

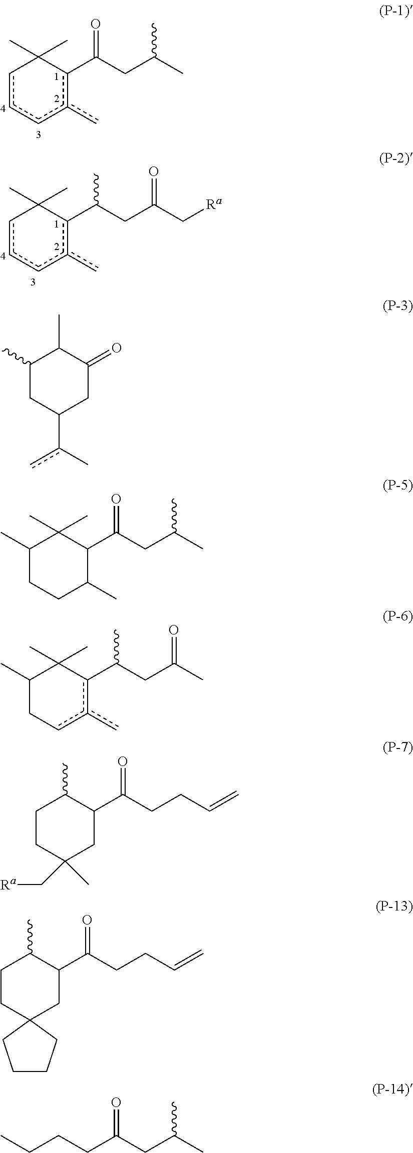

wherein the wavy lines have the meaning indicated above and the dotted lines represent a single or double bond, and Ra being a hydrogen atom or a methyl group.

In a particular embodiment, P represents a radical selected from the group consisting of formulae (P-1), (P-2), (P-1)′, (P-2)′, (P-3), (P-7), (P-13), (P-14) or (P-14)′ as defined above. Preferably, P represents a radical selected from the group consisting of formulae (P-1), (P-1)′, (P-2), (P-2)′, (P-3) or (P-14)′ as defined above.

In a particular embodiment, G may represent a divalent cyclic, linear, alicyclic or branched alkyl, alkenyl, alkandienyl or alkylbenzene hydrocarbon radical having from 1 to 22 carbon atoms, said hydrocarbon radical being possibly substituted and containing from 1 to 10 functional groups selected from the group consisting of ethers, esters, ketones, aldehydes, carboxylic acids, thiols, thioethers, amines, quaternary amines and amides.

In a particular embodiment, G represents a divalent linear or branched alkyl or alkenyl hydrocarbon radical having from 1 to 22 carbon atoms, said hydrocarbon radical being possibly substituted and containing from 1 to 5 functional groups selected from the group consisting of ethers, esters, ketones, aldehydes, carboxylic acids, thiols, thioethers, amines, quaternary amines and amides.

In a particular embodiment, G represents a divalent linear or branched alkyl or alkenyl hydrocarbon radical having from 2 to 15 carbon atoms, said hydrocarbon radical being possibly substituted and containing from 1 to 2 functional groups selected from the group consisting of ethers and esters.

In a particular embodiment, G represents a divalent linear alkyl or alkenyl hydrocarbon radical having from 3 to 15 carbon atoms, said hydrocarbon radical being possibly substituted and containing one ester functional group.

In a particular embodiment, G represents a divalent linear alkyl or alkenylhydrocarbon radical having from 3 to 14 carbon atoms.

In a particular embodiment, Q represents a hydrogen atom or a co-polymer comprising at least one repeating unit of formula B-1 as defined above.

In a particular embodiment, Q represents a hydrogen atom or a co-polymer comprising at least one repeating unit of formula B-1 and at least one repeating unit of formula B-2.

In a particular embodiment, R⁷ represents, simultaneously or independently, a hydrogen atom or a C₁₋₃ alkyl group. Preferably, R⁷ represents, simultaneously or independently, a hydrogen atom or a methyl or an ethyl group. More preferably, R⁷ represents, simultaneously or independently, a hydrogen atom or a methyl group.

In a particular embodiment, the first pro-perfume compound is defined by formula (II) as mentioned above, wherein
w represents an interger from 1 to 5000;
n=1; m=1;
P represents a radical susceptible of generating an odoriferous α,β-unsaturated ketone, aldehyde and is represented by the formula

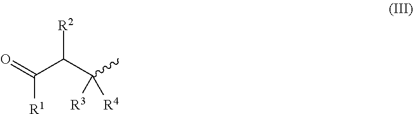

wherein R², R³ and R⁴ represent independently of each other a hydrogen atom, a C₆ to C₁₀ aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups;
G represents a divalent radical derived from cyclic, linear or branched alkyl, alkenyl, phenyl, alkylphenyl or alkenylphenyl hydrocarbon radical having from 2 to 8 carbon atoms optionally comprising 1 or 2 oxygen, sulfur and/or nitrogen atoms
Q represents a polymer or random co-polymer derived from formula B-1), wherein R⁷ represents a C₁-C₁₆ hydrocarbon group.

In a particular embodiment, the sulfur-containing pro-perfume compound is a linear polysiloxane co-polymer comprising at least one repeating unit of formula

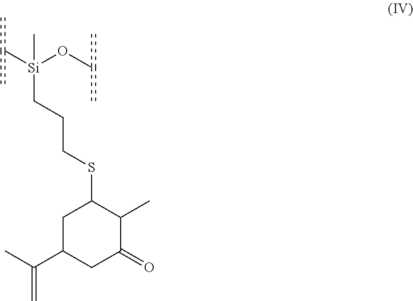

wherein the double hatched lines indicate the bonding to another repeating unit.

The pro-perfume of formula (IV) releases 2-methyl-5-(prop-1-en-2-yl)cyclohex-2-en-1-one as fragrance compound, which is also known as carvone. Carvone exists in the form of two enantiomers, namely (R)-(−)-2-methyl-5-(1-propen-2-yl)-2-cyclohexen-1-one (1-carvone or carvone laevo) and (S)-(+)-2-methyl-5-(1-propen-2-yl)-2-cyclohexen-1-one (d-carvone or carvone dextro). The two enantiomers have been reported to have slightly different mint odor tonalities. Nevertheless, according to the invention, both enantiomers are expected to have a similar effect in view of the preparation of the co-polymer and the release efficiency. According to the invention, carvone can either be used as a racemate or as a mixture enriched in either one of the two enantiomers. Preferably, a mixture enriched in carvone laevo is used.

In a particular embodiment, the sulfur-containing pro-perfume compound is of formula (V)

P-S-G-Q (V)

### Wherein

P has the same meaning as defined above:
G represents a divalent radical derived from a linear or branched alkyl or alkenyl radical having from 2 to 15 carbon atoms, possibly substituted with one or more groups selected from the group consisting of —OR⁹, —NR⁹₂, —COOR⁹ and R⁹ groups, in which R⁹ represents a hydrogen atom or a C₁ to C₆ alkyl or alkenyl group; and
Q represents a hydrogen atom.

According to more specific embodiments of component c), the pro-fragrance chemical is a formula (II) compound wherein P and Q have the same meaning as defined above, G is a divalent radical derived from a linear or branched alkyl or alkenyl radical having from 2 to 15 carbon atoms, possibly substituted by a —COOR⁹ group, wherein R⁹ is defined as above. More preferably, G is a divalent radical derived from a linear alkyl radical having from 8 to 15 carbon atoms or a —CH₂CH(COOR⁹) group, wherein R⁹ is a hydrogen atom or a methyl or ethyl group.

According to more specific embodiments of component c), the pro-fragrance is a compound selected from the group consisting of formulae (i) to (iv)

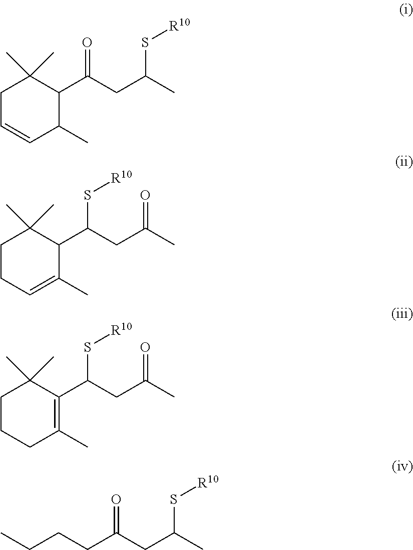

wherein R¹⁰ represents a C₁-C₂₀ alkyl or alkenyl group, preferably a C₆-C₁₆ alkyl or alkenyl group, more preferably a C₁₂ alkyl group.

The pro-perfume of formula (i) releases delta-damascone as fragrance compound. Said pro-perfume may preferably be (±)-trans-3-(dodecylthio)-1-(2,6,6-trimethyl-3-cyclohexen-1-yl)-1-butanone. Delta-damascone is also known as 1-[(1RS,2SR)-2,6,6-trimethyl-3-cyclohexen-1-yl]-2-buten-1-one.

The pro-perfume of formula (ii) or (iii) releases ionone as fragrance compound. Said pro-perfume may be present as an isomeric mixture of formula (ii) and formula (iii). The isomeric mixture may have a weight ratio of formula (ii) and formula (iii) from 40:60 to 60:40. In particular, the isomeric mixture may have a weight ratio of formula (ii) and formula (iii) of about 55:45. In particular, said pro-perfume releases two isomers of ionone as fragrance compound.

In particular, the pro-perfume of formula (ii) releases alpha-ionone as fragrance compound. Said pro-perfume of formula (ii) may preferably be (±)-4-(dodecylthio)-4-(2,6,6-trimethyl-2-cyclohexen-1-yl)-2-butanone. Alpha-ionone is also known as (±)-(3e)-4-(2,6,6-trimethyl-2-cyclohexen-1-yl)-3-buten-2-one.

In particular, the pro-perfume of formula (iii) releases beta-ionone as fragrance compound. Said pro-perfume of formula c) may preferably be (±)-4-(dodecylthio)-4-(2,6,6-trimethyl-1-cyclohexen-1-yl)-2-butanone. Beta-ionone is also known as (3e)-4-(2,6,6-trimethyl-1-cyclohexen-1-yl)-3-buten-2-one.

The pro-perfume of formula (iv) releases oct-2-en-4-one as fragrance compound. Said pro-perfume may preferably be (±)-2-(dodecylthio)octan-4-one. Oct-2-en-4-one may be released as its (E)- or (Z)-isomers, or as mixtures thereof, with the (E)-isomer being preferred.

According to any above embodiments, the at least one sulfur-containing pro-fragrance compound may be selected from the group consisting of methyl or ethyl 2-(4-oxo-4-(2,6,6-trimethylcyclohex-3-en-1-yl)butan-2-ylamino)-3-(4-oxo-4-(2,6,6-trimethylcyclohex-3-en-1-yl)butan-2-ylthio)propanate, methyl or ethyl 2-(4-oxo-4-(2,6,6-trimethylcyclohex-2-en-1-yl)butan-2-ylamino)-3-(4-oxo-4-(2,6,6-trimethylcyclohex-2-en-1-yl)butan-2-ylthio)propanate, methyl or ethyl 2-(2-oxo-4-(2,6,6-trimethylcyclohex-1-en-1-yl)butan-4-ylamino)-3-(2-oxo-4-(2,6,6-trimethylcyclohex-1-en-1-yl)butan-4-ylthio)propanate, methyl or ethyl 2-(2-oxo-4-(2,6,6-trimethylcyclohex-2-en-1-yl)butan-4-ylamino)-3-(2-oxo-4-(2,6,6-trimethylcyclohex-2-en-1-yl)butan-4-ylthio)propanate, 3-(dodecylthio)-1-(2,6,6-trimethylcyclohex-3-en-1-yl)-1-butanone, 3-(dodecylthio)-1-(2,6,6-trimethylcyclohex-2-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 2-dodecylsulfanyl-5-methyl-heptan-4-one, 2-cyclohexyl-1-dodecylsulfanyl-hept-6-en-3-one, 3-(dodecylthio)-5-isopropenyl-2-methylcyclohexanone, 2-(dodecylthio)-4-octanone, 4-(dodecylthio)-4-methylpentan-2-one, methyl or ethyl N,S-bis(4-oxo-4-(2,6,6-trimethylcyclohex-3-en-1-yl)butan-2-yl)-L-cysteinate, methyl or ethyl S-(4-oxo-4-(2,6,6-trimethylcyclohex-3-en-1-yl)butan-2-yl)-L-cysteinate, 4-oxooctan-2-yl dodecanoate, and any mixtures thereof, all other three components a), b) and d) being defined as previously, according to anyone of their possible embodiments cited, have proved most advantageous for the purposes of the invention. Particularly, the at least one sulfur-containing pro-fragrance compound may be selected from the group consisting of 3-(dodecylthio)-1-(2,6,6-trimethylcyclohex-3-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 3-(dodecylthio)-5-isopropenyl-2-methylcyclohexanone, 2-(dodecylthio)-4-octanone and a mixture thereof.

Perfumed consumer product comprising 3-(dodecylthio)-1-(2,6,6-trimethylcyclohex-3-en-1-yl)-1-butanone as the pro-fragrance component proved to be most advantageous.

The concentrations in which component c) is used in the perfumed consumer product of the invention are of at least 0.0001% by weight, of the weight of the composition. Particularly, the concentration of sulfur-containing pro-fragrance compound in the perfumed consumer product may be comprised between 0.001 and 5% based on the total weight of the perfumed consumer product. Particularly, the concentration of sulfur-containing pro-fragrance compound in the perfumed consumer product may be comprised between 0.001 and 2% based on the total weight of the perfumed consumer product. Particularly, the concentration of the sulfur-containing pro-fragrance compound in the perfumed consumer product may be comprised between 0.01 and 1% based on the total weight of the perfumed consumer product. Particularly, the concentration of the sulfur-containing pro-fragrance compound in the perfumed consumer product may be comprised between 0.01 and 0.5% based on the total weight of the perfumed consumer product. Even more particularly, the concentration of the sulfur-containing pro-fragrance compound in the perfumed consumer product may be comprised between 0.01 and 0.2% based on the total weight of the perfumed consumer product.

The component d) of the composition is one or more perfuming co-ingredient. By “perfuming co-ingredient” it is meant here a compound, which is used in a perfuming preparation or a composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin.

In particular one may cite perfuming co-ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0-2,7-]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients:methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

Component d) according to the invention may not be limited to the above mentioned perfuming co-ingredients, and many other of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds also known as properfume or profragrance. Non-limiting examples of suitable properfume may include 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene(2-((2-pentylcyclopentylidene)methoxy)ethyl)benzene or a mixture thereof.

The different ingredients of the invention's perfumed consumer product, in particular components c) and d), may be used in a pure form or in combination with a carrier of current use in perfumery. So, According to any embodiments of the invention, the component c) and/or d) of the composition may further comprise a perfumery carrier. By “perfumery carrier” it is meant here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethano, tri-ethyl citrate or mixtures thereof, which are the most commonly used. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. Solid carriers are of current use in the art and a person skilled in the art knows how to reach the desired effect. However by way of non-limiting examples of solid carriers, one may cite absorbing gums or polymers or inorganic materials, such as porous polymers, cyclodextrins, wood based materials, organic or inorganic gels, clays, gypsum talc or zeolites.

As other non-limiting examples of solid carriers, one may cite encapsulating materials.

Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof.

Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine based resins with aldehydes includes articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, vol. 7, pages 8041-8054.

According to any embodiments of the invention, the component d) of the composition may comprise a perfumery adjuvant. By “perfumery adjuvant”, it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof.

According to any embodiment of the perfumed consumer product of the invention, the latter contain at least 0.01% of at least one of such perfuming co-ingredients, and up to 3% weight, relative to the total weight of the composition. Preferred concentrations of the perfuming co-ingredients are comprised between 0.1 and 2 weight % and more preferably between 0.2 and 1.8 weight %, of the total weight of the liquid composition.

It has been established that the combination of components entering into the compositions here-described provides surprisingly improved fabric treatment products, over prior known products of similar type, their odor quality out of the bottle being remarkably more pleasant and stable, without any off-odor formation, and the odor of surfaces, in particular fabrics, treated with such compositions being perceived as longer lasting and more fresh.

The perfumed consumer products of the invention are useful in methods of treatment of various surfaces, in particular skins, hairs, fabrics and textiles. In such methods of use, they shall be applied as is current in washing and other fabric treating methods, both manually and in machine washing procedures, to produce their perfuming and long-lasting odor effect that is desired to impart to such fabrics.

Such use methods are also the object of the invention.

The invention is now described in further detail by way of the following examples, wherein the abbreviations have the usual meaning in the art. The Zinc salt used are commercially available and the pro-fragrance compounds c) used were prepared by the methods generally described in the prior art documents U.S. Pat. No. 7,723,286, WO 2014/202591 and/or WO 2008/154765.

### EXAMPLES

The invention will now be described in further detail by way of the following examples, wherein the abbreviations have the usual meaning in the art, the temperatures are indicated in degrees centigrade (° C.).

### Example 1

### Quantification of H₂S in a Fabric Softener Base Comprising Different Zinc Salts

The perfumed consumer product comprising at least one sulfur-containing profragrance compound may suffer from odor stability issue which may be due to the generation of H₂S having a detrimental impact on the odor.

Different zinc salts have been tested to evaluated their scavenging efficiency in a fabric softener base with the following composition:

| | |
|---|---|
| Stepantex ® VL90 A (origin: Stepan) | 12.21% by weight |
| Calcium chloride (10% aq. solution) | 0.40% by weight |
| Water | 87.39% by weight |

The amount of H₂S added to softener base with Zn Acetate at 0.2% and compared to the scavenging efficiency of Zinc bis[(9Z,12R)-12-hydroxy-9-octadecenoate] at 0.5% (comparative example), Sodium zinc polyitaconate at 0.5% (comparative example) and 1,2-benzisothiazolin-3-one at 0.5% (comparative example reported in WO2012113746), has been monitored over time using an analytical method based on in-fiber derivatization with N-ethylmaleimide combined with the high sensitivity and selectivity of the MS used in SIM mode as reported in ChemPlusChem 2014, 77.

### Apparatus and Method

The analysis has been performed, as reported in ChemPlusChem 2014, 77, using chromatography-mass spectrometry (GC-MS) (Shimadzu QP-2020, column DB-1MS 30.5 m, 0.25 mm, 0.25μ) in solid-phase microextraction (SPME) mode. The derivatization of H₂S was performed using a PAL autosampler, a 100% PDMS fiber was employed. The fiber was exposed to the headspace of the N-ethylmaleimide and triethylamine (NEM/TEA—400 μl of a 30/70 w/w solution of N-ethylmaleimide and triethylamine in a 20 ml headspace vial) reagent for 60 sec and then to that of the analyte for 1 min prior to desorption in the GC injector at 280° C.

The temperature program of the GC-MS was: 100° C. hold for 1 min; heat to 300° C. hold 2 min at 25° C./min and the acquisition has been performed in SIM mode, monitoring the masses 284, 158, 127.

The sum of the area of the two peaks related to the two pairs of diastereoisomers of (NEM)₂S resulted from the derivatization of H₂S has been used for the evaluation.

### Samples Preparation and Data Treatment:

In order to evaluate the scavenging efficiency of Zn salts, 2 g of softener base with Zn Acetate at 0.2% or Zinc Ricinoleate at 0.5% (comparative example) or Sodium zinc polyitaconate at 0.5% (comparative example) or 1,2-benzisothiazolin-3-one at 0.5% (comparative example reported in WO2012113746) and 2 g of softener base without scavenger were added in 6 autosampler vials and 0.6 ppm of H₂S was added to each vial before the analysis.

Each vial containing the softener base with H₂S and Zn salt has been injected after a vial containing the softener base with H₂S only and the % of loss of H₂S in presence of Zn salt for each data point was calculated as the difference to 100 of the ratio between the area of the peaks related to the derivatized H₂S in the two samples injected consecutively.

The evaluation of the scavenging efficiency over time have been performed over ˜6 h, injecting a vial every 33 min; 6 data points for each zinc have been acquired.

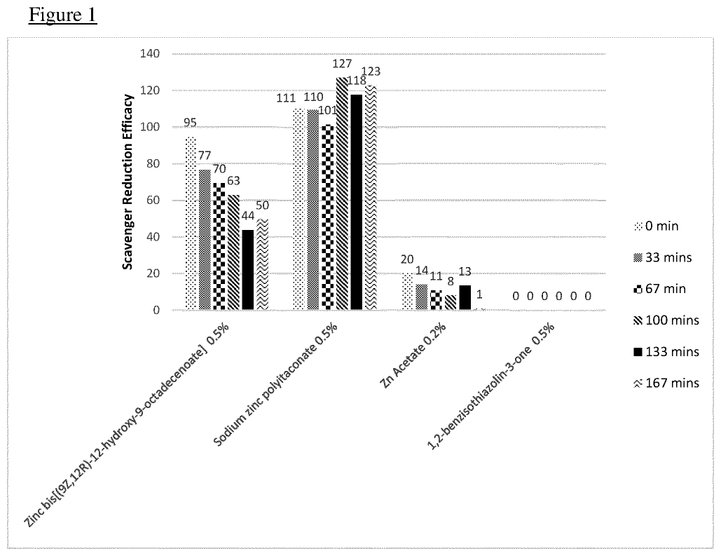

The results obtained are summarized in FIG. 1.

As shown in FIG. 1, the amount of H₂S was lower in the presence of Zinc acetate at 0.2% compared to Zinc bis[(9Z,12R)-12-hydroxy-9-octadecenoate] at 0.5%, or Sodium zinc polyitaconate. Zinc acetate allowed to obtain performance close to 1,2-benzisothiazolin-3-one which the use is limited in consumer product.

### Example 2

### Sensory Panel of the Invention's Perfumed Consumer Product—Fabric Softener

A typical perfume oil was prepared by admixing the following perfuming co-ingredients:

| Ingredients | weight-% |
|---|---|
| Ethyl 2-methylbutanoate | 0.16 |
| Hexyl acetate | 0.37 |
| Limonene | 1.67 |
| 2,6-Dimethyl-7-octen-2-ol | 0.94 |
| 2-Phenylethanol | 2.15 |
| Linalool | 0.73 |
| (2RS,4SR/4RS)-4-Methyl-2-(2-methyl-1-propen-1- | 0.30 |
| yl)tetrahydro-2H-pyran | |
| Ethyl 2-methyl-1,3-dioxolane-2-acetate | 0.32 |
| Benzyl acetate | 2.46 |
| Allyl heptanoate | 0.38 |
| alpha-Terpineol | 0.88 |
| 3,7-Dimethyl-6-octen-1-ol | 0.55 |
| 4-Methoxybenzaldehyde | 1.00 |
| (E)-4-Methyl-3-decen-5-ol | 0.37 |
| [cis/trans-4-(2-Propanyl)cyclohexyl]methanol | 0.47 |
| 1-Methoxy-4-[(1E)-1-propen-1-yl]benzene | 0.15 |
| (1RS,2RS/2SR)-2-(2-Methyl-2-propanyl)cyclohexyl | 1.95 |
| acetate | |
| 1,1-Dimethyl-2-phenylethyl acetate | 0.95 |
| Tricyclo[5.2.1.0^{2,}~]dec-3/4-en-8-yl acetate | 3.34 |
| Allyl 3-cyclohexylpropanoate | 0.26 |
| 3-(4-Isopropylphenyl)-2-methylpropanal | 8.18 |
| (3E)-3-Methyl-4-(2,6,6-trimethyl-2-cyclohexen-1- | 1.13 |
| yl)-3-buten-2-one and (1E)-1-(2,6,6-trimethyl-2- | |
| cyclohexen-1-yl)-1-penten-3-one | |
| 2-Phenoxyethyl 2-methylpropanoate | 5.38 |
| Tricyclo[5.2.1.0(2,6)]dec-3/4-en-8-yl propanoate | 2.32 |
| 5-Heptyldihydro-2(3H)-furanone | 2.30 |
| 2/3-Methylbutyl salicylate | 1.42 |
| (3Z)-3-Hexen-1-yl salicylate | 0.31 |
| 1-(2,3,8,8-Tetramethyl-1,3,4,5,6,7- | 16.03 |
| hexahydronaphthalen-2-yl)ethanone | |
| Hexyl 2-hydroxybenzoate | 5.04 |
| (2E)-2-Benzylideneoctanal | 21.22 |
| (−)-(3aR,5aS,9aS,9bR)-3a,6,6,9a- | 0.27 |
| Tetramethyldodecahydronaphtho[2,1-b]furan | |
| 1-Oxa-12/13-cyclohexadecen-2-one | 4.78 |
| Oxacyclohexadecan-2-one | 3.82 |
| Benzyl 2-hydroxybenzoate | 3.01 |
| Dipropylene glycol | 5.39 |
| | Total: 100 |

To this perfume oil, 2% of 3-(dodecylthio)-1-[(1RS,2SR)-2,6,6-trimethyl-3-cyclohexen-1-yl]-1-butanone was added. The perfume oil obtained was stored at 50° C. for two weeks. Aged Fragrance oil was added at defined dosage (1.8%) to the fabric softener base reported in example 1 comprising optionally 1,2-benzisothiazolin-3-one or zinc acetate as indicated in Table 1. The obtained mixture was stored for 4 weeks at 43° C. and then was evaluated by panelist (expert panel) at week 1, 2, 3 & 4. The panelist were asked to rate sample according to scale:
OK=no H2S odour detectable
S.MO=Slight H2S odour detectable
H₂S=noticeable H2S odour present.

The results obtained are summarized in Table 1.

TABLE 1
Panel evaluation in a fabric softener a.

| | Dosage | 1 wk/ | 1 wk/ | 2 wk/ | 3 wk/ | 4 wk/ |
|---|---|---|---|---|---|---|
| Scavenger | (%) | RT | 43° C. | 43° C. | 43° C. | 43° C. |
| 1,2- | 0.04 | OK | OK | OK | OK | OK |
| benzisothiazolin- | | | | | | |
| 3-one | | | | | | |
| Blank | — | H₂S | H₂S | H₂S | H₂S | H₂S S |
| Zn Acetate | 0.1 | OK | OK | OK | OK | OK |
| Zn Acetate | 0.15 | OK | OK | OK | OK | OK |

As clearly shown in the Table 3, Product comprising Zinc Acetate do not show any H₂S malodor contrary to the reference without any salts.

### Example 3

### Sensory Panel of the Invention's Perfumed Consumer Product—Transparent Isotropic Shampoo

A typical unperfumed transparent isotropic shampoo formulation is listed in Table 2. The unperfumed shampoo formulation was prepared by dispersing Polyquaternium-10 in water. The remaining ingredients of Phase A were mixed separately by addition of one after the other while mixing well after each adjunction. This pre-mix was added to the Polyquaternium-10 dispersion and mixed for another 5 min. Then, the premixed Phase B and the premixed Phase C were added (Monomuls® 90L-12 was heated to melt in Texapon® NSO IS) while agitating. Phase D and Phase E are added while agitating. The pH is adjusted with a citric acid solution to 5.5-6.0.

TABLE 2
Composition of a typical unperfumed transparent
isotropic shampoo formulation.

| | | Amount |
|---|---|---|
| Phase | Ingredients | [wt %] |
| A | Deionized water | 44.4 |
| | Polyquaternium-10 ⁽¹⁾ | 0.3 |
| | Glycerin 85% ⁽²⁾ | 1.0 |
| | DMDM Hydantoin ⁽³⁾ | 0.2 |
| B | Sodium laureth sulfate ⁽⁴⁾ | 28.0 |
| | Cocamidopropyl betaine ⁽⁵⁾ | 3.2 |
| | Disodium cocoamphodiacetate ⁽⁶⁾ | 4.0 |
| | Ethoxy (20) stearyl alcohol ⁽⁷⁾ | 1.0 |
| C | Sodium laureth sulfate ⁽⁴⁾ | 3.0 |
| | Glyceryl laureate ⁽⁸⁾ | 0.2 |
| D | Deionized water | 1.0 |
| | Sodium methylparaben ⁽⁹⁾ | 0.1 |
| E | Sodium chloride (10% aqueous solution) | 15.0 |
| | Citric acid (10% aqueous solution to pH | q.s. |
| | 5.5-6.0) | |

⁽¹⁾ Ucare ® Polymer JR-400; origin: Noveon
⁽²⁾ Origin: Brenntag Schweizerhall AG
⁽³⁾ Glydant ®; origin: Lonza
⁽⁴⁾ Texapon ® NSO IS; origin: Cognis
⁽⁵⁾ Tego ® Betain F 50; origin: Evonik
⁽⁶⁾ Amphotensid GB 2009; origin: Zschimmer & Schwarz
⁽⁷⁾ Brij ® S20; origin: Croda
⁽⁸⁾ Monomuls ® 90 L-12; origin: Gruenau GmbH
⁽⁹⁾ Nipagin Monosodium; origin: NIPA

The perfumed shampoo formulation was then obtained by adding, under gentle shaking, a perfume oil reported in Table 3 (1% by weight relative to the total weight of the unperfumed shampoo formulation listed in Table 2), 3-(dodecylthio)-1-[(1RS,2SR)-2,6,6-trimethyl-3-cyclohexen-1-yl]-1-butanone (0.2% by weight relative to the total weight of the unperfumed shampoo formulation listed in Table 2) and triethanolamine (0.04% by weight relative to the total weight of the unperfumed shampoo formulation listed in Table 2). Then optionally a Zinc salt was added as indicated in Table 4 to obtain products A, B and C comprising respectively no Zinc salt, Zinc acetate and Zinc stearate.

TABLE 3
Composition of Perfume oil

| Ingredients | Qty (g) |
|---|---|
| Carbinyl acetate | 2.20 |
| Citronellyl acetate | 16.59 |
| Linalyl acetate | 10.72 |
| Nopyl acetate | 7.97 |
| Terpinyl acetate | 2.11 |
| Verdyl acetate | 2.89 |
| Decanal | 0.07 |
| Hexylcinnamic aldehyde | 13.94 |
| Ethyl 2-methyl-pentanoate ¹⁾ | 0.26 |
| Benzyl benzoate | 8.19 |
| Allyl (cyclohexyloxy)acetate | 2.14 |
| Hedione ® ²⁾ | 11.94 |
| Hexyl isobutyrate | 2.63 |
| 2-{2-[4-methyl-3-cyclohexen-1- | 10.34 |
| yl]propyl}cyclopentanone | |
| 2-methyl-4-propyl-1,3-oxathiane | 0.08 |
| Tetrahydro-4-methyl-2-(2-methyl-1-propenyl)-2h-pyran | 0.44 |
| Verdyl propionate | 4.33 |
| Beta ionone | 0.52 |
| (3Z)-3-hexen-1-yl (3Z)-3-hexenoate | 1.24 |
| 2,4-Dimethyl-3-cyclohexene-1-carbaldehyde ³⁾ | 1.42 |

¹⁾ Origin: Firmenich SA, Geneva, Switzerland
²⁾ Cyclopentaneacetic acid, 3-oxo-2-pentyl-, methyl ester; origin and trademark from Firmenich SA, Geneva, Switzerland
³⁾ Origin: Firmenich SA, Geneva, Switzerland

Product A, B and C were placed for 2 weeks in accelerated stability (RT vs 50° C.) and H₂S malodor was olfactively evaluated by expert panelists after 1 week and 2 weeks.

The results obtained are summarized in Table 4.

TABLE 4
Panel evaluation in a transparent isotropic shampoo.

| | | Dosage | 1 wk/ | 1 wk/ | 2 wk/ | 2 wk/ |
|---|---|---|---|---|---|---|
| Products | Scavengers | (%) | RT | 50° C. | RT | 50° C. |
| A | Reference | — | Medium | Strong | Medium | Strong |
| | without Zinc | | H₂S | H₂S | H₂S | H₂S |
| | Salts | | malodor | malodor | malodor | malodor |
| B | Zinc Acetate | 1% | OK | OK | OK | OK |
| C | Zinc Stearate | 0.5% | OK | OK | OK | OK |

As clearly shown in the Table 4, Product B & C of the invention (post addition of Zinc salts respectively 1% Zinc Acetate and 0.5% of Zinc Stearate) do not show any H₂S malodor like the reference without any salts.

1. A perfumed consumer product comprising:
a) an active base;
b) at least one Zinc salt of formula
Zn(RCOO)₂ (I)
wherein R is a hydrogen atom or a linear C₁-C₂₀ alkyl or linear C₂-C₂₀ alkenyl group;
c) at least one sulfur-containing pro-fragrance compound; and
d) one or more perfuming co-ingredients.

2. The perfumed consumer product according to claim 1, consisting of a mixture of components a) to d).

3. The perfumed consumer product according to claim 1, wherein component a) is a liquid softener active base, liquid detergent active base, shampoo active base or an all-purpose cleaner active base.

4. The perfumed consumer product according to claim 1, wherein the perfumed consumer product has a pH comprised between 1 and 8.

5. The perfumed consumer product according to claim 1, wherein component a) represents at least 85%, up to 99.95%, by weight, of the total weight of the perfumed consumer product.

6. The perfumed consumer product according to claim 1, wherein R is a hydrogen atom or a linear C₁-C₁₈ alkyl group.

7. The perfumed consumer product according to claim 6, wherein component b) is Zinc acetate or Zinc stearate.

8. The perfumed consumer product according to claim 6, wherein component b) is present at a concentration of 0.01 weight % or more, and can form up to 5% weight, of the total weight of the perfumed consumer product.

9. The perfumed consumer product according to claim 1, wherein the at least one sulfur-containing pro-fragrance compound is at least one compound of formula 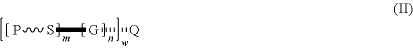
wherein:
a) w represents an integer from 1 to 10000;
b) n represents 1 or 0;
c) m represents an integer from 1 to 6;
d) P represents a hydrogen atom or a radical susceptible of generating an odoriferous α,β-unsaturated ketone, aldehyde or carboxylic ester and is represented by the formula 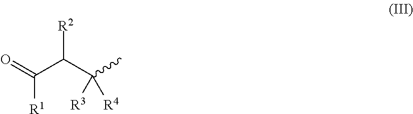
in which the wavy line indicates the location of the bond between said P and S;
R¹ represents a hydrogen atom, a C₁ to C₆ alkoxyl radical or a C₁ to C₁₅ linear, cyclic or branched alkyl, alkenyl or alkadienyl radical, optionally substituted by one to four C₁ to C₄ alkyl groups; and
R², R³ and R⁴ represent independently of each other a hydrogen atom, an aromatic ring, or a C₁ to C₁₅ linear, cyclic or branched alkyl, alkenyl or alkadienyl radical, possibly substituted by C₁ to C₄ alkyl groups; or two, or three, of the groups R¹ to R⁴ are bonded together to form a saturated or unsaturated ring having 5 to 20 carbon atoms and including the carbon atom to which said R¹, R², R³ or R⁴ groups are bonded, this ring being possibly substituted by C₁ to C₈ linear, branched or cyclic alkyl or alkenyl groups; and with the proviso that at least one of the P groups is of the formula (III) as defined hereinabove;
e) G represents a multivalent radical (with a m+1 valence) derived from cyclic, linear, alicyclic or branched alkyl, cyclic, linear, alicyclic or branched alkenyl, phenyl, alkylphenyl or alkenylphenyl hydrocarbon radical having from 1 to 22 carbon atoms, said hydrocarbon radical being possibly substituted and containing from 1 to 10 functional groups selected from the group consisting of halogens, alcohols, ethers, esters, ketones, aldehydes, carboxylic acids, thiols, thioethers, amines, quaternary amines and amides; and
f) Q represents a hydrogen atom (in which case w=1 and n=1), or represents a polymer or co-polymer selected from the group consisting of poly(alkylimine)s, peptides (e.g. lysine) or polysaccharides selected from the group consisting of cellulose, cyclodextrins and starches, or cationic quaternised silicon polymers, or still a polymer or random co-polymer derived from monomeric units selected from the group consisting of the formulae A) to C): 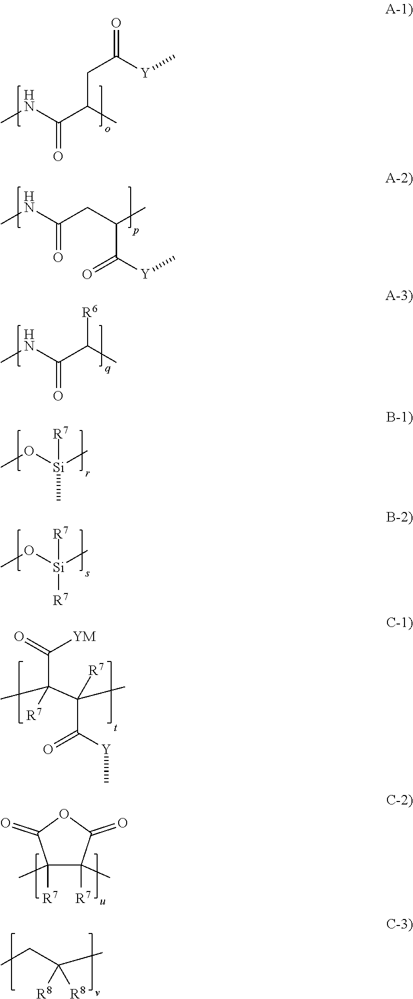
wherein the hatched lines indicate the location of the bond between said monomeric unit and G;
Y represents an oxygen or sulfur atom or a NR⁷ group;
o, p, q, r, s, t, u and v all represent independent of each other fractions between 0 and 1, with o+p+q=1, r+s=1 and t+u+v=1 and with the proviso that either o or p, as well as r and t are not equal to 0;
R⁶ represents a hydrogen atom or a side chain from a natural or unnatural amino acid, such as glycine, alanine, phenylalanine, arginine, histidine, lysine, aspartic acid, glutamic acid, cysteine, methionine, glutamine, asparagine, threonine, serine, leucine, isoleucine, valine, tyrosine or tryptophan;
R⁷ represents, simultaneously or independently, a hydrogen atom or a C₁-C₁₆ hydrocarbon group;
R⁸ represents, simultaneously or independently of each other
a hydrogen or halide atom;
a C₁-C₆ hydrocarbon group optionally comprising from 1 to 4 heteroatoms selected from the group consisting of oxygen and sulfur atoms;
a carboxylic group of formula COOR*, wherein R* represents a hydrogen atom, a C₁-C₆₀ alkyl or alkenyl group optionally comprising from 1 to 30 oxygen atoms;
a OR⁷ group or a COR⁷ group; or
a pyrrolidone unit, connected by the nitrogen atom; and
M represents a hydrogen atom, an alkali or earth alkali metal ion.

10. The perfumed consumer product according to claim 9, wherein the at least one sulfur-containing pro-fragrance compound is a compound of formula (II) wherein G is a divalent radical derived from a linear or branched alkyl or alkenyl radical having from 2 to 15 carbon atoms, optionally substituted with a —COOR⁹ group, R⁹ being a hydrogen atom or a C₁ to C₆ alkyl or alkenyl group.

11. The perfumed consumer product according to claim 1, wherein the at least one sulfur-containing pro-fragrance compound is a compound of formula (II) wherein P is a radical selected from the group consisting of formulae (P-1) to (P-14), in the form of any one of its isomers: 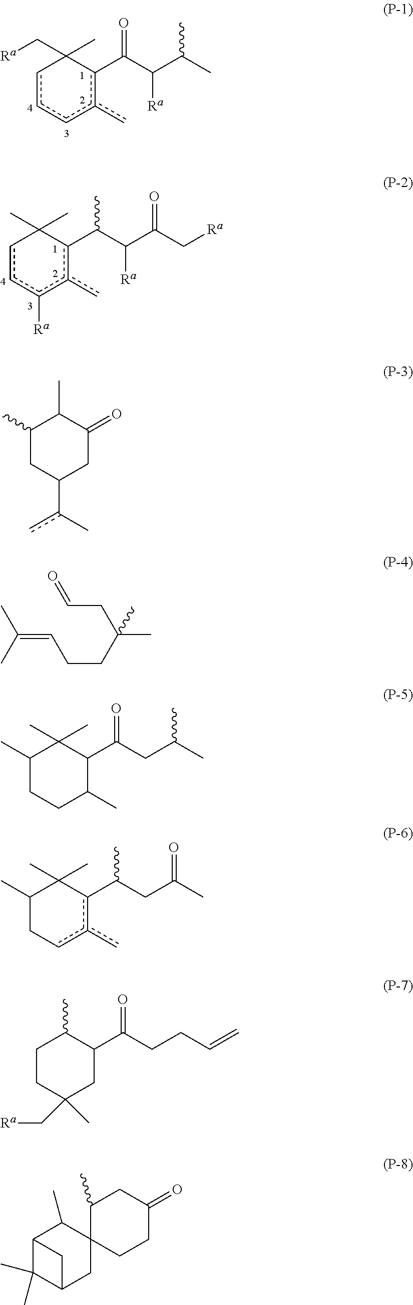 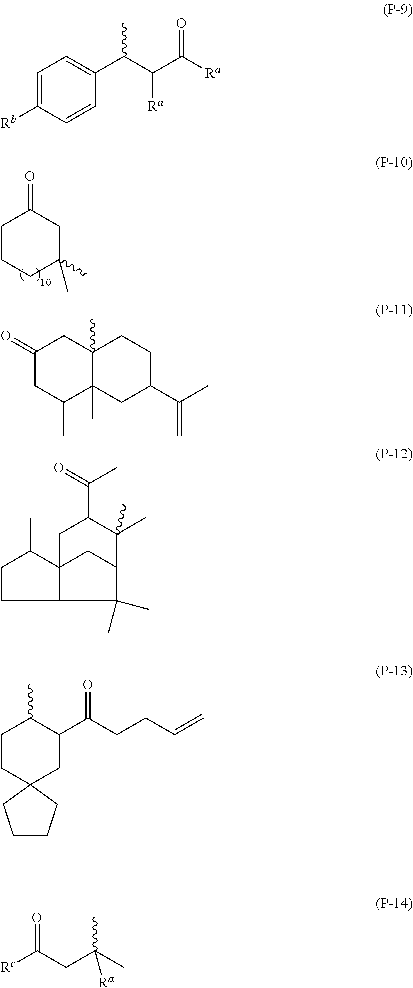
wherein the wavy line indicates the location of the bond between said P and S; the dotted lines is a single or double bond; Ra is a hydrogen atom or a methyl group; R^{b} is a hydrogen atom, a hydroxyl or methoxy group or a C₁-C₄ linear or branched alkyl group and R^{c} is a hydrogen atom or a C₁-C₄ linear or branched alkyl group.

12. The perfumed consumer product according to claim 9, wherein the at least one sulfur-containing pro-fragrance compound is selected from the group consisting of methyl or ethyl 2-(4-oxo-4-(2,6,6-trimethylcyclohex-3-en-1-yl)butan-2-ylamino)-3-(4-oxo-4-(2,6,6-trimethylcyclohex-3-en-1-yl)butan-2-ylthio)propanate, methyl or ethyl 2-(4-oxo-4-(2,6,6-trimethylcyclohex-2-en-1-yl)butan-2-ylamino)-3-(4-oxo-4-(2,6,6-trimethylcyclohex-2-en-1-yl)butan-2-ylthio)propanate, methyl or ethyl 2-(2-oxo-4-(2,6,6-trimethylcyclohex-1-en-1-yl)butan-4-ylamino)-3-(2-oxo-4-(2,6,6-trimethylcyclohex-1-en-1-yl)butan-4-ylthio)propanate, methyl or ethyl 2-(2-oxo-4-(2,6,6-trimethylcyclohex-2-en-1-yl)butan-4-ylamino)-3-(2-oxo-4-(2,6,6-trimethylcyclohex-2-en-1-yl)butan-4-ylthio)propanate, 3-(dodecylthio)-1-(2,6,6-trimethylcyclohex-3-en-1-yl)-1-butanone, 3-(dodecylthio)-1-(2,6,6-trimethylcyclohex-2-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 2-dodecylsulfanyl-5-methyl-heptan-4-one, 2-cyclohexyl-1-dodecylsulfanyl-hept-6-en-3-one, 3-(dodecylthio)-5-isopropenyl-2-methylcyclohexanone, 2-(dodecylthio)-4-octanone, 4-(dodecylthio)-4-methylpentan-2-one, methyl or ethyl N,S-bis(4-oxo-4-(2,6,6-trimethylcyclohex-3-en-1-yl)butan-2-yl)-L-cysteinate, methyl or ethyl S-(4-oxo-4-(2,6,6-trimethylcyclohex-3-en-1-yl)butan-2-yl)-L-cysteinate, 4-oxooctan-2-yl dodecanoate, and any mixtures thereof.

13. The perfumed consumer product according to claim 12, wherein the at least one sulfur-containing pro-fragrance compound is selected from the group consisting of 3-(dodecylthio)-1-(2,6,6-trimethylcyclohex-3-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 3-(dodecylthio)-5-isopropenyl-2-methylcyclohexanone, 2-(dodecylthio)-4-octanone and a mixture thereof.

14. The perfumed consumer product according to claim 9, wherein the at least one sulfur-containing pro-fragrance compound is present in at least 0.0001 weight % of the total weight of the perfumed consumer product.

15. Method for the treatment of a surfaces, the method comprising contacting the surface with a perfumed consumer product according to claim 1.

16. The method according to claim 15, wherein the surface is a body, hair or a home fabric.

17. The perfumed consumer product according to claim 1, wherein:
component a), present in an amount from 85% to 99.95%, by weight of the total weight of the perfumed consumer product, is a liquid softener active base, a liquid detergent active base, a shampoo active base or an all-purpose cleaner active base;
component b), present in an amount from 0.01% to 5% by weight of the total weight of the perfumed consumer product, is Zinc acetate or Zinc stearate; and
the at least one sulfur-containing pro-fragrance compound, present in an amount of at least 0.0001% by weight of the total weight of the perfumed consumer product, is selected from the group consisting of 3-(dodecylthio)-1-(2,6,6-trimethylcyclohex-3-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 3-(dodecylthio)-5-isopropenyl-2-methylcyclohexanone, 2-(dodecylthio)-4-octanone and a mixture thereof.

18. Method for the washing and/or softening of a body, hair or home fabric, the method comprising treating the body, hair or home fabric with a perfumed consumer product according to claim 17.

19. The perfumed consumer product according to claim 2, wherein:
component a), present in an amount from 85% to 99.95%, by weight of the total weight of the perfumed consumer product, is a liquid softener active base, a liquid detergent active base, a shampoo active base or an all-purpose cleaner active base;
component b), present in an amount from 0.01% to 5% by weight of the total weight of the perfumed consumer product, is Zinc acetate or Zinc stearate; and
the at least one sulfur-containing pro-fragrance compound, present in an amount of at least 0.0001% by weight of the total weight of the perfumed consumer product, is selected from the group consisting of 3-(dodecylthio)-1-(2,6,6-trimethylcyclohex-3-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 3-(dodecylthio)-5-isopropenyl-2-methylcyclohexanone, 2-(dodecylthio)-4-octanone and a mixture thereof.
